(19) United States
(12) Patent Application Publication
(10) Pub. No.: US 2003/0130567 A1
(43) Pub. Date: Jul. 10, 2003

(54) Health-related devices and methods

(76) Inventors: James R. Mault, Evergreen, CO (US); Thomas Kilbourn, Saratoga, CA (US); Christopher L. Sandys, Evergreen, CO (US); Geoffrey G. Moyer, Portola Valley, CA (US)

Correspondence Address: Allen M. Krass, Gifford, Krass, Groh, Sprinkle,, Anderson & Citkowski, P.C., 280 N. Old Woodward Ave., Suite 400, Birmingham, MI, 48009-5394, US

(21) Appl. No.: 10/338,391

(22) Filed: Jan. 8, 2003

Related U.S. Application Data
(60) Provisional application No. 60/347,134, filed on Jan. 9, 2002.
(60) Provisional application No. 60/362,030, filed on Mar. 6, 2002.

(51) Int. Cl. A61B 5/00

(52) U.S. Cl. 600/300

(57) ABSTRACT

A device, comprises an electrical circuit, comprising a processor, a memory, and a clock, operable to determine a physiological parameter of a user from a physiological signal; a counter, having a count number decremented by an operation of the electrical circuit to determine the physiological parameter; and a software program, executable by the processor, operable to receive an authorization code, to determine an increment number from the authorization code, and to change the count number by the increment number, wherein the device can be used a greater number of times by the user after receipt of the authorization code.

### REFERENCE TO RELATED APPLICATIONS

This application claims priority from U.S. provisional application Serial Nos. 60/347,134, filed Jan. 9, 2002 and 60/362,030, filed Mar. 6, 2002, the content of each application being incorporated herein by reference.

### FIELD OF THE INVENTION

Embodiments of the present invention relate to improved devices and methods, in particular the prepayment of use of medical test equipment.

### BACKGROUND OF THE INVENTION

The home use of devices, such as medical equipment, is of great value to many people. However, a business supplying such equipment may not want to sell the equipment outright to a person. For the purposes of regulatory approval, it may be highly advantageous to closely monitor the use of the equipment, providing updates as required.

Conventional methods of prepayment for services include the use of magnetic cards for photocopiers, postal machines, and the like. However, the prior art does not describe the provision of medical equipment to a person, whereby the person can buy a predetermined number of uses of the equipment over a communications network.

Also, the provision of healthcare within a medical environment such as a hospital can include a series of problems with billing and expense tracking. It is highly advantageous to provide a system which allows the use of equipment to be tracked and conveniently billed to the correct entity.

In U.S. Pat. No. 6,282,522, Davis et al. describe an Internet payment system using a smart card. However, Davis et al. do not disclose devices having an internal counter.

In U.S. Pat. No. 5,878,141, Daly et al. describe a computerized purchasing system for mediating purchase transactions over a network. However, this patent does not disclose the prepayment of services provided by devices having an internal counter, the internal counter being decremented upon each use.

Patents and patent applications (including provisional patent applications, U.S. applications, and international applications) mentioned within this specification are incorporated herein by reference. U.S. Pat. Nos. 4,917,108; 5,038,792; 5,178,155; 5,179,958; 5,836,300; 6,135,107; 6,277,645; 6,309,360; 6,402,698 and 6,468,222 (to Mault) disclose respiratory analyzers such as indirect calorimeters which may be advantageously adapted for use as devices according to embodiments of the present invention. Additional approaches to indirect calorimetry, cardiac output monitoring, and health-related services to a user are disclosed in co-pending applications Ser. Nos. 09/721,382, 09/008,435; 09/191,782; PCT/US99/02448; PCT/US99/17553; PCT/US99/27297; and PCT/US00/12745.

Physiological monitoring systems, methods, and devices, which may be used in embodiments of the present invention, are described in Int. App. Nos. WO01/89368, WO01/82789, WO01/52718, WO01/39089, WO01/28416, and published U.S. application Ser. Nos. US20,010,049,470 and US20,010,044,588.

Calorie management systems and methods, elements of which may be used in embodiments of the present invention, are described in International App. Nos. WO01/89365, WO01/82783, WO01/28495, and WO01/26535, U.S. application Ser. No. 09/721,382, and U.S. Pat. No. 6,478,736.

### SUMMARY OF THE INVENTION

Embodiments of the present invention include methods and systems for facilitating the use of a device, the device being used provide an operation of value to a user. (A user is a subject using the device). For example, the device may provide medical services within a hospital, doctor's office, clinic, fitness center, the user's home, or other location. The device may comprise the functionality of a physiological monitor (such as a metabolic rate meter such as an indirect calorimeter).

Hence, a device according to an embodiment of the present invention, comprises an electrical circuit, comprising a processor, a memory, and a clock, operable to determine a physiological parameter of a user, such a metabolic parameter (such as metabolic rate, oxygen consumption, or carbon dioxide production) from a physiological signal (such as oxygen concentration, flow rate, carbon dioxide concentration, heart rate, body temperature, EKG, or other physiological parameter); a counter, having a count number decremented by an operation of the electrical circuit to determine the physiological parameter; and a software program, executable by the processor, operable to receive an authorization code, to determine an increment number from the authorization code, and to change the count number by the increment number, wherein the device can be used a greater number of times by the user after receipt of the authorization code. The software program can be further operable to prevent operation of the device if the count number is less than a predetermined value, such as one or zero.

The device can further comprises a data communications device, such as a modem, data port, wireless transceiver, or the like, providing a communications links with an electronic apparatus, wherein the authorization code is received from the electronic apparatus. The electronic apparatus may comprises a computer, telephone, (such as a wireless phone), entertainment device (such as an Internet appliance or television), or the like. A data communications device, associated with the device, may provide a communications link with a communications network, wherein the authorization code is received from a remote computer over the communications network. A user of the device may enter an authorization code into the device using a data input mechanism, such as a keypad, voice recognition system, or other data entry mechanism

A device, having the functionality of a respiratory analyzer (such as an indirect calorimeter), may comprise a flow path, through which a subject breathes; a respiratory gas analyzer, providing gas analysis data at least containing flow rate data correlated with a flow rate of respired gases through the flow path; electrical circuitry, comprising a processor, a memory, and a clock, receiving the gas analysis data and operable to provide a determination of a metabolic parameter of the subject; a data input mechanism (such as a keypad, data port, data transceiver, link to a communications network, modem, and the like), operable to receive an authorization code; and a software program, executable by the processor, operable to receive the authorization code, and to enable operation of the device a number of times, the number of times being contained (e.g. encrypted or encoded) within the authorization code. For example, the authorization code may be generated algorithmically using a serial number, the increment number, and a random number. The device may comprises a flow rate meter, oxygen sensor, and/or a carbon dioxide sensor.

An improved system for providing a service to a subject comprises a device, comprising a processor and a memory, operable to determine a health-related parameter of the subject, such as a metabolic parameter or other physiological parameter; a communications link between the device and a computer; computer software, running on the computer, operable to receive identifying data either from the device, the user of the device, the user's insurer, or other entity, to generate an authorization code, and to transmit the authorization code to the device over the communications link; and device software, executed by the processor within the device, operable to receive the authorization code from the computer over the communications link, and to enable one or more operations of the device after receipt of the authorization code. Memory may comprise RAM, ROM, and other forms of volatile or non-volatile memory. The computer may be a remote computer associated with a health services provider.

A method for obtaining at least one operation of a medical device, the medical device providing a health-related service to a subject (such as determination of a metabolic parameter), comprises: providing an identifier to a supplier of the medical device; receiving an authorization code from the supplier of the medical device; providing the authorization code to the medical device; and operating the respiratory analyzer a number of times, the number of times being encoded within the authorization code, and the number of times being at least one.

The identifier can be a serial number of the device, user name or other user identifier, insurance policy number, or other identifier. Providing the identifier to the supplier of the medical device may comprise transmitting the identifier to a computer associated with the supplier of the medical device over a communications network, and receiving the authorization code may comprise receiving the authorization code over a communications network, such as the Internet or other public network. The device may comprise software adapted to decrypting the authorization code so as to determine an increment number; incrementing a count number associated with the medical device by the increment number; decrementing the count number on each successful operation of the device; and disabling the device if the count number falls below a predetermined value.

A method of obtaining a health-related service using a medical device (such as an indirect calorimeter or other physical parameter determination device), comprises: purchasing an authorization code; providing the authorization code to the medical device, wherein a count number associated with the medical device is incremented by an increment number contained by the authorization code; operating the medical device, so as to obtain the health-related service, wherein the count number is decremented each time the medical device is operated, and wherein the device does not operate after the count number falls to a predetermined value.

Purchasing an authorization code can comprise: communicating with a remote computer over a communications network; providing an identifier to the computer; and receiving the authorization code from the computer over the communications network. Financial information, such as a credit card number, may also be provided to the seller of the authorization code, health-related service provider, insurance company, or the like. Providing the authorization code to the medical device comprises may include entering the code into a numeric keypad in communication with the medical device, connecting to another electronic apparatus (which may be connected to a communications network, or otherwise connecting the device to a communications network. Providing the authorization code to the medical device may also comprises storing the authorization code on a token, such as a smart card, and providing the token to a token reader in communication with the medical device.

In one embodiment, the device comprises a counter, a processor, a memory, a clock, an operational module, and a data entry mechanism. The data entry mechanism may comprise a serial port, parallel port, wireless transceiver (such as Bluetooth, wireless Ethernet, IEEE 801(b) or other wireless device), IR sensor, or other transceiver. The counter may be provided by software or hardware. The counter has an associated counter number, for example an integer stored in memory. The operational module provides the operation of value, for example a measurement of a person's metabolic rate. After each successful operation, the counter number is decremented by one (or other number). If the counter number decreases to be equal to or less than a predetermined value, the device becomes inoperable. Provision of an authorization code to the device allows the counter number to be incremented by an increment number, allowing the device to be operated.

A system including a device may further comprise an electronic apparatus (such as a computer) in communication with the device over a communications link between the electronic apparatus and the device. For example, the device may be in communication with a personal computer. The personal computer can further be in communication with a remote server computer over a communications network. Software on the server computer can provide an authorization code over the communications network.

In one embodiment, a server software program, executed by a processor within the server computer, is operable to generate an authorization code, and transmit the authorization code to the device over the communications link, either directly or using an electronic apparatus in communication with communications network and the device. A device software program, executed by the processor within the device, is operable to determine an increment number from the authorization code, to increment a counter number by the increment number, and to decrement the counter when the device is successfully operated. The counter number can be stored within the device, for example in memory, or stored in another location in communication with the device.

A communications link between the device and an electronic apparatus (such as a personal computer), between the electronic apparatus and a server computer, or between the device and the server computer, may comprise or be provided, at least in part, by a cable, wireless communications link, satellite link, communications network, telephone network, IR or optical link, human intervention (for example, reading or otherwise sensing information from one location and providing it to another), or other data communication mechanism.

A device, providing a useful operation (such as a metabolic rate measurement) can comprise an operational module (such as a module corresponding in functionality to an indirect calorimeter) providing a success signal on performance of a successful operation performed to a predetermined success standard, and providing an error signal on performance of an unsuccessful operation, a counter, responsive to successful operations; a processor; a software program executable by the processor, operable to receive an authorization code, to determine an increment number from the authorization code, to increment the counter by the increment number; to decrement the counter on receipt of a test signal.

The device may further comprise a data input mechanism, wherein the authorization code can be entered using the data input mechanism. The device can further comprise a communications link to a communications network, and the authorization code can be received over the communications network, for example from a remote server system. The device can further be provided with a communications link to an electronic apparatus in possession of a user of the device. For example, the electronic apparatus may be a computing device (such as a personal digital assistant (PDA)), telephone (such as a wireless telephone), entertainment device (such as a television, interactive television, Internet appliance, docking station (discussed in more detail below) or other electronic apparatus.

A business may provide a person with a device according to embodiments of the present invention. It may not be advantageous to either the business or the user for the device to be purchased outright by the user. Lease arrangements are possible, but these do not account for the number of times a device is used. Hence, embodiments of the present invention advantageously allow a user to be charged based on usage of a device.

### BRIEF DESCRIPTION OF THE DRAWINGS

FIG. 1 shows a device having a counter and operational module connected over a communications network to a computer;

FIG. 2 shows a device in communication with an electronic apparatus, the electronic apparatus being in communication with a server computer over a communications network;

FIG. 3 shows an alternative configuration of the system of FIG. 2;

FIG. 4 illustrates a system in which an authorization code can be received on an electronic apparatus;

FIG. 5 illustrates a method of operating a device according to an embodiment of the present invention; and

FIGS. 6 and 7 illustrate other system embodiments in which an authorization code is provided over a communication network.

### DETAILED DESCRIPTION OF THE PREFERRED EMBODIMENTS

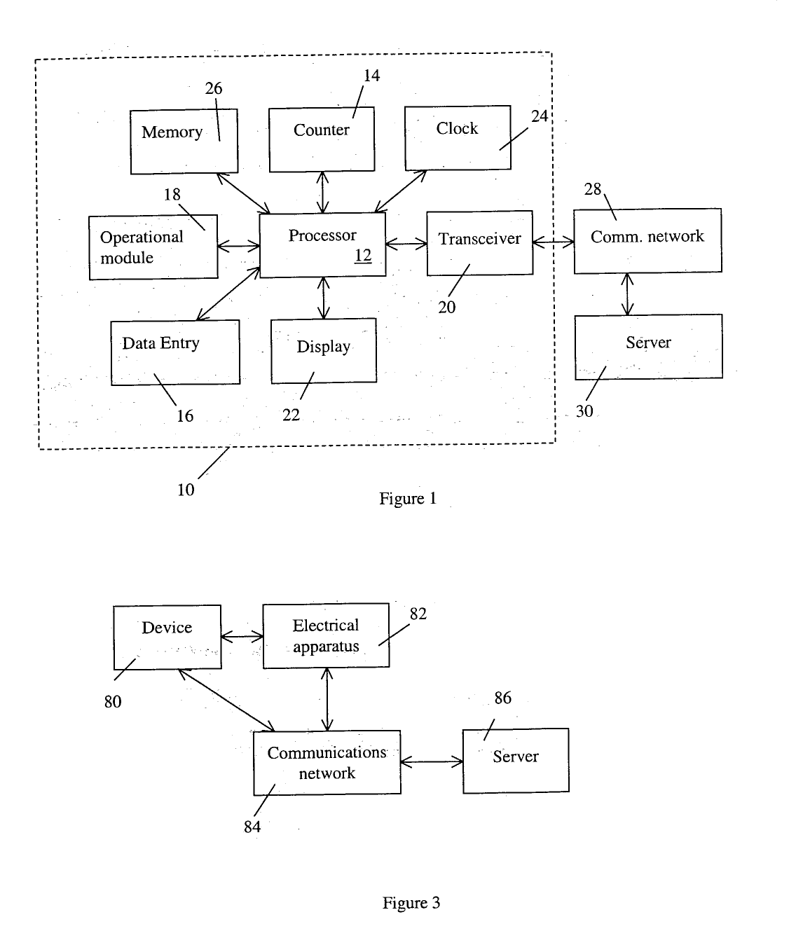

FIG. 1 shows a device shown generally at 10 comprising processor 12, counter 14, data entry mechanism 16, operational module 18, transceiver 20, display 22, clock 24, memory 26, in communication with communications network 28, and server computer 30.

The operational module is operable to assist the provision of a useful operation. For example, the useful operation may be the provision of health-related services such as the determination of one or more physiological parameters (such as metabolic rate, blood glucose level, EKG, or some other physiological parameter). The device may provide a medical test, medical service, or other operation.

For example, the device can be operable to determine an oxygen consumption of a subject. In this case, the operational module can includes electronic circuitry, transducers, and/or sensors so as to determine the composition of gases respired by a subject, so that device 10 has the functionality of an indirect calorimeter, for example as described in our co-pending applications and patents.

In this example, the operational module may comprise ultrasonic transducers and associated circuitry for the measurement of flow rates through a flow tube, gas component sensors and associated circuitry for the monitoring of gas component concentrations with respired gases, the operational module and other circuitry of device 10 operating together so as to provide an estimation of metabolic rate or some other respiratory parameter, such as oxygen consumption, carbon dioxide production, and the like. The processor 12 may be part of the circuitry allowing determination of metabolic rate or respiratory parameters.

The counter 14 is associated with a count number which is decremented on each successful use of device 10. For example, oxygen consumption measurements may be made using device 10 and an associated error code generated if the test is unsuccessful (for example, the test results or conditions do not conform with predetermined parameters). For example, an unsuccessful test may be inferred from an excessive error level, data having characteristics associated with test failure, uneven baselines, excessive fluctuations in one or more physiological parameters, or other test parameters. Conversely, a success code can be generated if the test is successful (for example, the test results or conditions do conform with predetermined parameters). The count number may only be decremented by successful tests. In other embodiments, some error codes may cause the count number to be decremented, for example those associated with inappropriate user behavior.

Device 10 includes a counter 14 and a software program executable by processor 12 which prevents operation of the device if the count number associated with counter 14 is below a predetermined number, such as zero. In order to operate the device 10, the user supplies an authorization code to the device. The authorization code may be encrypted, for example using an encryption algorithm which generates the authorization code using, for example, the increment number, a serial number associated with the device, and a random number. Decryption software on the device extracts the serial number and increment number from the authorization code. The count number is incremented by the increment number if the serial number matches that of the device. In other embodiments, only a serial number or other identifier is encrypted in the authorization code, and the count number is incremented by a predetermined value on receipt of the authorization code. The authorization code may contain check digits for self-authentication.

A user can connect the device 10 to the communications network 24 and so to the server computer 30. An authorization code is sent from the server computer through the communications network to the device. This process may happen automatically, or may require the user initialization in some manner, for example by sending a request message, calling a phone number, pressing a button, keys, or icon on the device 10, or some other method such as interacting with the data entry mechanism 16.

After the device receives the authorization code, a software program executable by processor 12 (device software) decrypts the authorization code so as to extract an increment number. The increment number is then added to the count number, allowing the device to be used an additional number of times, correlated with the increment number.

The device may send an identifier to the server computer, for example a serial number, or some identifier relating to the entity owing the device, which may be a person, organization or other group. The server computer may have access to a database containing financial information relating to the user of device 10. Prior to sending the authorization code, or after sending the code, or simultaneously, a server software program may cause a corresponding billing or charge against a financial account associated with the user. This is an advantageous method of payment for use of device 10.

Data entry mechanism 16 may be one or more buttons or keys, a voice recognition system, a touch pad, or other mechanism. The transceiver 20 may be a wireless modem, other wireless transceiver, or other device. The display 22 may be a liquid crystal display, light emitting diode display, or similar. The memory 26 may comprise volatile memory, nonvolatile memory, ROM, RAM, EEPROM, removable memory modules, or any combination of memory components. The counter 14 may be executed in hardware or software. The counter has an associated counter number, which may be stored within a separate counter device, or within the memory 26, for example within an EEPROM or protected memory. A decryption key, used by device software to decrypt the authorization code, may also be stored in a memory location.

The transceiver 20 and communications network 24 constitute, at least in part, a communications link between the device 10 and the server computer 30. In other embodiments the communications link between the device 10 and server computer 30 may comprise a telephone line, electronic apparatus in communications with the device 10 and the communications network, a satellite link, an optical link, a cable link, human activity (such as reading data over a telephone, reading data from a web site, entering data into the data input mechanism), modem, wireless modem, local network, other wide area network, or other communications link or method.

The server computer may comprise one or more processors, server software application program executable on a processor, memory, a database, and the like. The database may be used to store financial information, health information, nutrition information, and the like relating to the user of the device. The server computer may also provide a visual representation of data stored within the database, for example in the form of a web page. The server software program may be operable to receive an identifier from a user of the device, and to bill an entity associated with the identifier in return for providing the authorization code.

The system described in relation to FIG. 1 allows the supplier of the device to charge for each use of the device. The system also allows the supplier to track usage and collect statistics relating to the use of the device. These may be transmitted over the communications network to the server computer, and stored in a database. The system allows a user to be charged per use of the device, and not on the basis of the cost of the device. Also, device software upgrades can be transmitted from the server computer to the device when the device is connected. This may be advantageous in relation to regulatory approval of medical devices. The system necessitates the periodic communication between the device and the server computer, if the person is going to continue to use the device. Each time the device is connected, data can be transmitted from the device to the server computer and data can be transmitted from the server computer to the device. Exchanged information may comprise software upgrades, test usage statistics, financial information, other health-related factors which may be collected using the device, revised financial information, and the like.

If the device requires accessories for use, these may be shipped to the person when the authorization code is purchased. For example, in the case of an indirect calorimeter, the purchase of an authorization code enabling the device to be used a certain number of times may trigger the supply of the corresponding numbers of filters, mouthpieces, masks, or other required disposable accessories. Alternatively, the same authorization code may be used to enable the device to be used a predetermined number of times, and also to obtain a corresponding number of disposable accessories.

The display 22 may be used to display the results of device operation, for example a metabolic rate determination, financial information transmitted to or received from the server computer, the count number (for example the number of operations of the device that are allowed), error codes or other indications of failed device operation, and the like.

An audible alert or other non-visual indication may be used to indicate each successful use of the device.

The device, electronic apparatus in communication with the device, server computer, or other computer in communication with one or other system components, may comprise a database. The database can be used to store, for example, identification information, health records, body weight data relating to one or more possible users of the device, data trends over time, insurance information, or financial information (for example, related to an operator of the device).

Software (for example server software, device software, or software running on an other electronic apparatus such as a personal computer) can be operable to charge the operator in relation to the provision of an authorization code, for example by debiting a bank account, fitness center account, credit card, or other service account. The charge can be correlated with the increment number corresponding to the authorization code. Software can also be operable to receive an identifier from a user of the device (for example an account code, name, e-mail address, other alphanumeric code, bar code, biometric identifier (such as fingerprint or retinal pattern, or voice), or other identifier), and to bill an entity associated with the identifier (such as the user of the system, employer, company, health plan (such as HMO)) in relation to provision the authorization code.

The device may require a consumable in relation to its operation, for example hygiene components such as masks, pathogen filters, skin contacts, or other disposable or sterilizable components. Provision of such consumables can be correlated with provision of the authorization code.

Software on the device or electronic apparatus in communication with the device can be operable to receive the authorization code over the communications network, and can be further operable to receive a software update over the communications network, for example after purchase of an authorization code.

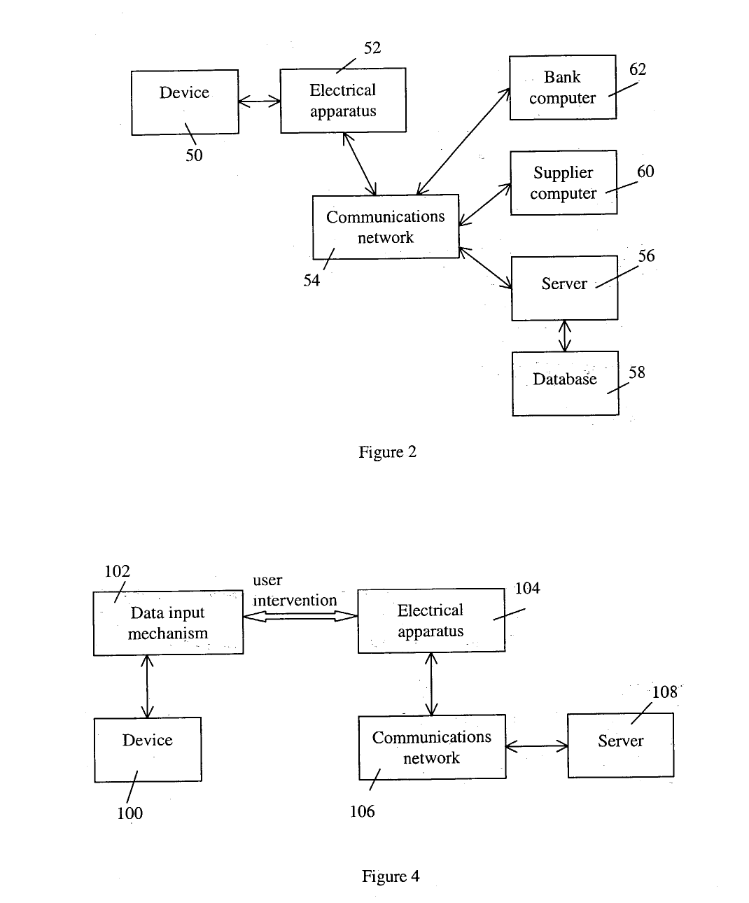

FIG. 2 shows an alternative configuration of a system according to the present invention. The system comprises device 50, electronic apparatus 52, communications network 54, server computer 56, database 58, supplier computer 60, and bank computer 62. The double-headed arrows represent communications links. Device 50 has a communications link with electronic apparatus 52, which is in communication with computers 56, 60, and 62 through the communications network 54. In other embodiments, device 50 may also have a direct communications link with the communications network that does not pass through the electronic apparatus 52.

The server computer may have server software operable to generate an authorization code, and to transmit the code to the electronic apparatus. The database may contain financial, health, or other information relating to the user of the device, and data relating to the device and supplier.

The supplier computer may be associated with a supplier of consumables related to operation of the device, for example masks, hygiene barriers, disposable components, and the like. Provision of an authorization code correlated with a certain number of operations of the device may cause a corresponding number of consumables to be shipped or otherwise provided to the user.

Software updates may also be provided to the user. Charges can be billed to the user's account, for example by communication with the bank computer. The user may provide authorization to charge a certain cost corresponding to the authorization code request.

For example, the device 50 may be an indirect calorimeter, and the electronic apparatus 52 may be a personal computer, personal digital assistant, wireless phone, chronometer, Internet appliance, interactive television, entertainment device or other apparatus having a connection to the communications network. The communications network may be the Internet, other wide area network, local network (for example within a hospital or fitness center), other telecommunications network, other wireless network, or other network.

The device user may install software, provided by the device manufacturer, on the electronic apparatus, for example on a personal computer. The user can then connect the device 50 to the electronic apparatus 52, for example using a serial cable, optical link, other wireless communications link, cradle, or other mechanism. The user then requests an authorization code from the device supplier using the software on the electronic apparatus. The user may enter payment information, or financial information relating to the user may be stored on the device, electronic apparatus, or server computer. Payment information, if supplied, can be transmitted to the server computer, supplier computer, or bank computer. An encrypted authorization code can then be transmitted to the electronic apparatus 52, and subsequently to the device 50, incrementing a counter number and allowing additional uses of the device. The user may then detach the device from the computer and exit the software.

In another embodiment, the electronic apparatus comprises a dial-up modem. The user installs software on the electronic apparatus, and then connects the device to the electronic apparatus, for example using a serial cable. The software then dials the server computer, so as to connect the electronic apparatus to the server computer. The user may also provide payment information, which is then also transmitted over the communications network to the server computer. An authorization code is then transmitted from the server computer to the electronic apparatus, and then provided to the device. In other embodiments, the supplier computer may be contacted instead, or the supplier computer and server computer may be the same computer, or the bank computer omitted from the system.

FIG. 3 shows a system configuration comprising electronic apparatus 82, device 80, communications network 84, and server computer 86. In this system configuration, the device comprises a communications link to a communications network 84 and a communications link to electronic apparatus 82. For example, the device may comprise a wireless transceiver having a communications link with the Internet. Alternatively, the device may have a modem capable of dialing a remote computer over a telephone network. The electronic apparatus may comprise a more sophisticated data entry mechanism, which may be used to enter authorization codes or financial information or other health-related data into the device for transmission to the server computer 86. The server may be in communication with a database.

FIG. 4 shows another system configuration comprising device 100, data input mechanism 102, electronic apparatus 104, communications network 106, and server computer 108. Using this system configuration, the user uses electronic apparatus 104 to receive an authorization code from the server computer. The authorization code is then input into the device using the input mechanism 102. The input mechanism may be, for example, an alphanumeric keypad, a voice recognition system, or other mechanism. For example, a numeric keypad may be attached to the device using a cable, and an authorization code input by typing.

For example, a user may use an electronic apparatus comprising a web browser to view a web page generated by a server computer. The user may enter a device serial number, personal identifier (which may be associated with a device serial number and other data on a database), and payment information. An authorization code is then displayed on the web page. The authorization code can then be entered into the device. The device may have keys, buttons, touch screen, touch pad, roller ball, voice recognition, or other data entry mechanisms. An external keypad may be connected to the device to allow the authorization code to be entered.

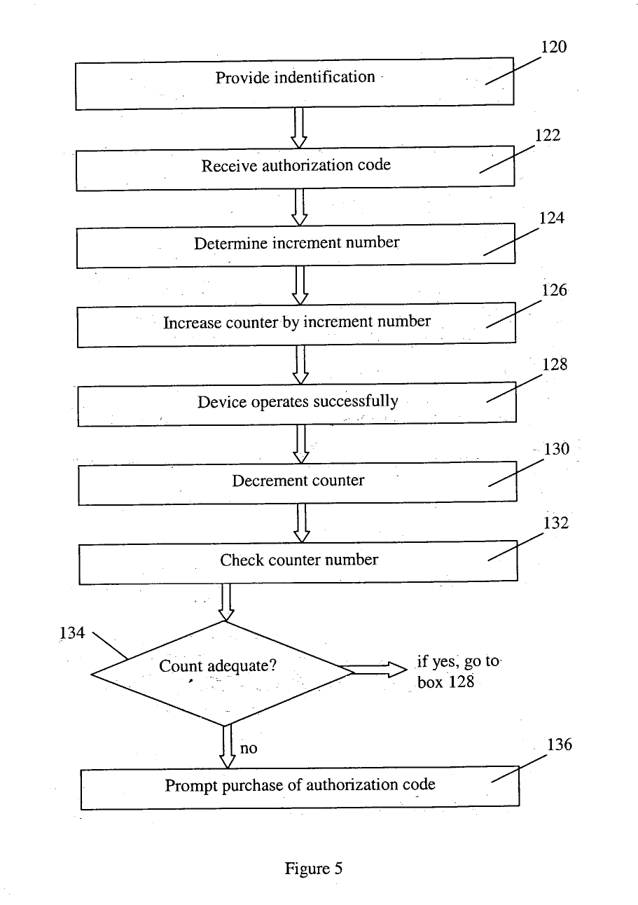

FIG. 5 shows a method of using an improved device according to the present invention. The user provides an identifier (box 120), which may be a personal identifier, a business identifier, serial number of the device, or similar. The identifier is transmitted to a remote computer. Box 122 corresponds to receiving the authorization code from the remote computer. The code may be received by the person, by the device, or provided to both.

Box 124 corresponds to determination of an increment number determined by the authorization code. For example, this may be a decryption step. Box 126 corresponds to incrementing the counter number by the increment number. For example, a person may purchase 100 uses of a device. An authorization code is provided to the person containing encrypted data comprising an increment number of 100. The counter number may be a certain value before receipt of the authorization code, so that subsequently the counter number is the previous number increased by 100. This allows the person to use the device an additional 100 times. Box 128 corresponds to use of the device. Box 130 corresponds to decrementing the counter after use of the device. The counter may only be decremented if the device operation is successful. In this context, successful operation may require test parameters to fall within predetermined ranges. In the example of oxygen consumption measurements, it may be necessary for respiration frequency, pulse rate, or other physiological or respiratory parameters to remain within predetermined limits over the duration of the test.

Box 132 corresponds to checking the counter, in particular determining whether the count number is greater than a predetermined level such as zero. Box 134 corresponds to e.g. testing whether the count number is greater than zero. If the count number is greater than zero, allowing further operation of the device, the user may proceed to step 128, device operation. If the counter is not greater than zero, the user may be prompted to obtain a further authorization code (box 136). This may require returning to the first step in which an identifier is provided. However, the identifier may be stored within the device memory, so that all the user has to do to receive a further authorization code is to respond positively through a prompt, press a button, connect the device to a communications network, connect the device to another electronic apparatus having a communications link to a communications network, and the like.

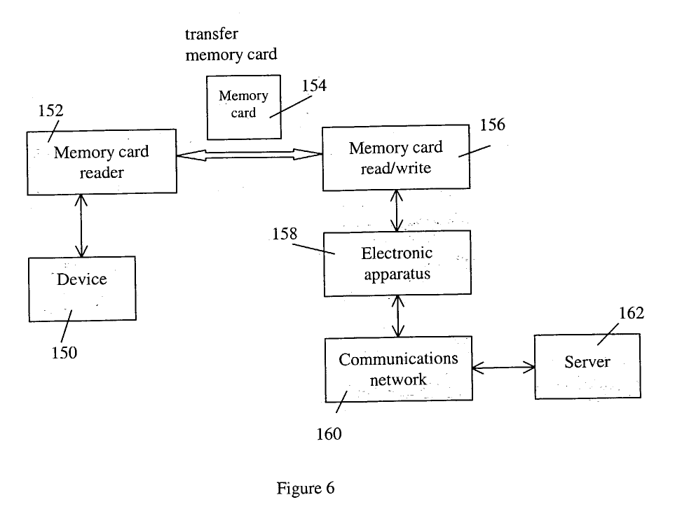

FIG. 6 shows another system embodiment comprising device 150, memory card reader 152, memory card 154, card read/write device 156, electronic apparatus 158, communications network 160, and server computer 162. In this system embodiment, the user provides a memory card 154 along with payment information, or personal identification, at the read/write device 156 connected to (or part of) the electronic apparatus 158. The memory card 154 receives an authorization code from the server computer over the communications network. The person then inserts the memory card 154 into the card reader 152 attached to the device 150. The card reader 152 receives the authorization code from the memory card, incrementing an internal counter within the device, and so allowing the device to be used a certain additional number of times. The device may have a memory in which previous authorization codes are stored, so that the same authorization code cannot be used more than once. The card reader 152 may have also a write function or a data deletion function, so as to erase or invalidate the authorization code once it has been read from the memory card. Other tokens (discussed in more detail below) can be used in place of the memory card.

For example, this system may be useful if the person has limited access to a computer having an Internet connection. The electronic apparatus 158 may be provided at a health center, doctor's office, recreation center, or similar location. Payment information may be stored on the memory card, and read by the card read/write device 156. Personal identification data may also be stored on the card 154. Test results obtained by the device, other physiological data, and the like may also be written to the card 154, and subsequently transmitted to the server computer 162. In another embodiment, the person is provided with a smart card at a store in return for a purchase price.

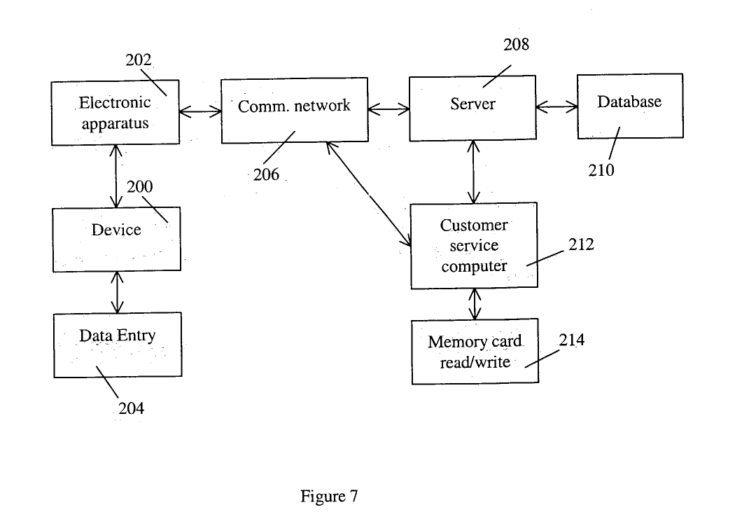

FIG. 7 shows another system embodiment, comprising a device 200, an electronic apparatus 202, a data entry mechanism 204, communications network 206, server computer 208, database 210, business customer service computer 212, and business smart card read-write device 214. The data entry mechanism may be a keypad or smart card reader accessible by the user. The server computer 208 and/or computer 212 may support authorization code generating software, and the electronic apparatus 202 or device 200 may support authorization code decryption software.

In one method of operation, the user calls or otherwise contacts (for example, using e-mail or a web site form) a customer service representative and provides identification information, such as name, social security number, e-mail address, password, credit card number, account number, device serial number, or other identification. The customer service representative verifies the identification information, and provides an authorization code, for example by reading out over the phone, on a personalized password-protected web site, through e-mail, other communication with the electronic apparatus or the device (such as by providing a sequence of audio tones or other encoded data). The user may enter the data through the data entry mechanism, for example by typing into a keypad.

The customer service representative may also mail a smart card or printed authorization code to the user. The above services may be automated by software running on the server computer 208 or computer 212.

The device may be medical test equipment, other test equipment (such as automotive test equipment), or the like. Embodiments of the present invention may be used in connection with viewing entertainment, vending machines, restaurant meals, purchase of utilities, gas purchase, motel room rental, mail machines, tickets, photocopies, product rental, parking, and the like. The authorization code may be an electronic code stored in computer memory. An authorization code may also be provided by a printed ticket, barcode, magnetic stripe, sticker, magnetic pattern, other pattern, discernable visual indication, hologram, or other method.

Embodiments of the present invention can be used in improved health improvement programs, such as weight loss programs. For example, a user may be provided with an indirect calorimeter and a diet logging device adapted to record consumable (for example, food) intake and activity expenditure, and adapted to calculated calorie balance. The user can purchase uses of the indirect calorimeter through purchase of an authorization code, as discussed above. The authorization code may be supplied over the communications network from a server. Health-related data relating to the user (such as body weight, diet information, or resting metabolic rate) may be transmitted to the server over the communications network. Feedback may be provided to the user over the communications network. If the resting metabolic rate of the user is changing, or the user has changed food consumption habits, the user may be encouraged to measure their resting metabolic rate more frequently, for example through the provision of authorization codes allowing additional uses of the indirect calorimeter.

In other embodiments of the present invention, the device may transmit information relating to the use of the device, and data collected by the device, to a server or other location. The shipment of consumables, such as masks or mouthpieces for use with an indirect calorimeter, may occur along with the provision of an authorization code.

If the user owns a computer with a connection to the Internet, the user can install software (for example, software supplied by the device supplier) on the computer, connect the device to the computer via a cable, wireless, socket, or other link or interface, request an authorization code using the software, and enter payment information, which is transmitted securely to a server associated with the device supplier. Device usage data can also be transmitted to the server. An encrypted authorization code is then transmitted to the computer, then to the device. The user may disconnect the device from the computer before using it, if convenient. An authorization code may enable a plurality of uses, or a single use.

If the user owns a computer with a dial-up modem, but does not have an Internet connection, the user can install the software on the computer, connect the device to the computer, and request an authorization code using the software. The software dials a server or other location associated with the device supplier. The user enters payment information, which is transmitted to the device supplier, and an encrypted authorization code is transmitted to the computer from the supplier, and so to the device.

A memory card can also be used, for example if the user has periodic access to a computer with a connection to the Internet. A memory card reader/writer is attached to the computer, and the user inserts a memory card into the reader/writer. The user requests an authorization code using software on the computer, and enters payment information. The payment information is transmitted to a server or other location associated with the device supplier. An encrypted authorization code is transmitted to the computer, then to the memory card. Memory cards can also be purchased through conventional retail channels, for example empty or pre-loaded at retail price. The user may enters personal data via the web and enters a serial number on the smart card, so that the memory card and information is now linked to the end user. The user can take the memory card to a service provider, for example a fitness center providing use an indirect calorimeter, and requests a test. The service provider uses the user's memory card to authorize the test. The serial number of the card is associated with the test just performed and logged on the device The next time the service provider connects the device to the device supplier (to a server via Internet, or using a smart card, etc.), the service provider can get credit for the test performed and can collect a fee.

Authorization codes can be used to purchase at least one use of a device at any location, including commercial locations, for example at a fitness center or other health related location. The user may store the authorization code in any convenient manner, and provide it when requesting use of a device. The user may subsequently be provided with an authorization code containing (e.g. having encrypted or encoded within) a lower increment number.

The user may also purchase a point-of-sale device comprising a memory card reader and a dial-up modem. The user may own a computer with a connection to the Internet, in which case the user can install software on the computer, for example to communicate with a server associated with the device supplier. The user inserts a smart card into the reader/writer, requests an authorization code using the software, or keypad, or other method, and enters payment information which is transmitted to a server or other location associated with the device supplier. An authorization code is transmitted to the user.

In other embodiments, the device may interact with a memory card reader, credit card reader. The device may have a direct wireless connection with a communications network. The device may be a unitary device, or may comprise a plurality of elements, such an indirect calorimeter with a docking station (discussed below). Authorization codes may be stored within a tangible object, such as the tokens discussed below, and mailed to the user. Service representatives or employees of a business associated with the device supplier may supply the authorization code to the user. The device may be initially supplied with a memory card containing an authorization code. The device may have any data input device to receive the authorization code, such as a barcode reader or optical character recognition in conjunction with an imaging sensor. The authorization code may be carried by a device in the form of a key or other security device.

Other Embodiments Using Token

A person can be provided with a token to assist participation within a health-related program. The token may have the form factor of a card (such as a credit card, membership card, smart card, memory card, or identity card), key, key fob, button, body ornamentation, dongle, or other form. The token can be used to facilitate payment for goods and services, receiving goods and services paid for in advance, use equipment, gain entry to facilities, to identify the holder, store authorization codes, and the like. For example, the token can be used to provide identification information, to receive authorization codes, and to provide authorization codes to devices. Authorization codes, discussed in more detail above, and tokens can be used in similar methods and systems.

The token may comprise data storage capabilities, such as magnetic media (such as a magnetic strip), bar code, optical memory, electronic memory, or other memory or recording medium. The token may have wireless transmission capabilities, so as to transmit a signal identifying the person to a suitably adapted receiving device, or may have wireless transponder capabilities, so as to generate a wireless signal in response to a signal transmitted by another device or system.

The token functionality may also be incorporated within another device, such as one carried by the person, for example as part of a watch, portable telephone, portable computer (such as a PDA) or other device. The token may have the form of a card inserted into another device. The functionality of the token described herein may be produced by one or more other devices, such as a telephone or computer.

The token can be used to log into or otherwise identify the person to health-related systems or devices. These may include systems according to embodiments of the present invention or other devices such as physiological monitors, other medical devices, exercise machines, indirect calorimeters. The token can also be used to identify the person to health professionals, building access devices, or other persons or devices.

For example, a person can swipe (or otherwise pass a token through a token reader) or otherwise bring a token into communication with an electronic apparatus associated with an exercise machine. An electronic circuit associated with the exercise machine can provide exercise data, for example exercise parameters such as duration of exercise, start time, finish time, repetitions, intensity of exercise, exercise machine used, machine settings, resistance, nature of exercise, and the like, and other data such as time of day. The exercise data can be transmitted to a computer in communication with the exercise machine. For example, the exercise data can then be stored in a database or other memory structure associated with the identity of the user provided by the token. The exercise data can be transmitted to the token for storage in memory therein.

Exercise parameters can be correlated with calorimetric expenditure (or equivalently, VO₂) of the person, for example by having the person breath through an indirect calorimeter while exercising. Methods have been described in my previous applications, such as WO01/189365, incorporated herein by reference. The personal identity provided by the token can be used to select customized correlation parameters from a memory, and energy expenditure for the person can then be determined from exercise parameters using correlation parameters for that person.

An exercise machine may receive an identity for a person, for example from a token carried by the person, so as to determine energy expenditure for that person as the person exercises. Energy expenditure can be calculated in real time from exercise parameters using a correlation chosen for the person exercising, for example one based on previous measurements or based on the demographic or physical parameters of the person, such as age, height, weight, gender and the like. Energy expenditure can be shown to the person on a display as they exercise.

Hence, an improved fitness improvement method for a person comprises: determining a correlation for the person between parameters of an exercise and energy expenditure by the person; providing the person with an identity device; using the identity device to identify the person during a later exercise by the person; and using the correlation determined for the person to determine energy expenditure by the person during the later exercise. The parameters of an exercise may include repetitions, intensity, load, resistance, heart rate, distance covered, activity sensor signal, pedometer signal, and the like. The identity device may be a token, electronic apparatus, or other identity token as described in more detail below. Biometrics, such as fingerprints, voice, or retinal properties, can also be used to identify a person for the purpose of applying a personalized correlation between activity and energy expenditure.

In this example, a token is generally assumed to have a physical form, such as a card. However, the token may also take the form of electronic data, for example the authorization codes discussed in more detail above. An authorization code (or other electronic code) may authorize a person to some number (e.g. 20 uses) of an indirect calorimeter or other device. After the completion of a successful test, the authorization code can be modified so as to authorize 19 uses. For example, the code may be electronic data carried on a PDA in communication with an indirect calorimeter.

A PDA, watch, telephone, or other portable electronic apparatus can be used to allow the person to log into a system, for example through the transmission of an identity code. The portable device can contain authorization codes to use fitness center devices such as metabolic rate meters and the like, and may contain authorization for a certain number of prepaid uses of an indirect calorimeter. The person can receive further authorization codes (which may be referred to as credits) over a communications network.

A token may also take the form of an identification mechanism. A token reader receives identity data from the token, and then verifies that the identity information allows use of the indirect calorimeter. For example, the token reader may be in communication with a database having prepayment data, or authorization data (for example if the identity corresponds to a subscriber), so that only persons that have pre-paid, subscribed, or are otherwise authorized, are allowed to use a device associated with the token reader.

Prepayment of Indirect Calorimeter Usage

A token can also be used for prepayment of use of an indirect calorimeter, other metabolic rate meter, exercise machine, physiological monitor, medical equipment, consultation, or other device or service. The token may have a magnetic strip or other data storage capability, whereby authorization for a certain number of uses of an indirect calorimeter is stored. Each time the person uses an indirect calorimeter, the number of authorized uses is decreased by one. The indirect calorimeter may be provided with software by which the success of the measurement can be determined, so that the person is not charged for an unsuccessful use of the indirect calorimeter.

A person can subscribe to a health-related program, such as a weight loss program, and be issued a token. The program can include free or for-fee use of equipment and services, depending on the subscription level chosen. For example, use of exercise equipment can be free, whereas use of an indirect calorimeter can be for a fee (to cover the use of disposable elements such as masks, mouthpieces, filters, pathogen barriers, and the like). The person pre-pays a sum of money, allowing the person to use the indirect calorimeter a certain number of times, for example by authorizing the person to use a certain number of disposable elements, by providing a certain number of valid authorization codes (which can be invalidated after use), or by providing an authorization code allowing for a certain number of uses. A token can also be used to identify the person to a computer system interfaced to suitably adapted exercise equipment, and to other equipment such as an indirect calorimeter, so that the resting metabolic rate measured is stored in a database associated with the owner of the token, and exercises performed can be converted into energy expenditure according to a customized relationship e.g. between repetitions and energy expenditure, which can be found for a person by monitoring their metabolic rate during an exercise.

A person may access a web site, for example associated with a health-related business, and be provided with an authorization code for a certain number of uses of an indirect calorimeter. This may be part of a subscription program, a promotion, or in return for other payment method.

A person may be provided with an indirect calorimeter for home use. The indirect calorimeter can be adapted to require an authorization code for use. This code can be provided by a web site, e-mail, telephone recording, or other electronic apparatus connectable to a communications network (such as an Internet appliance or interactive television). Authorization codes may be entered into the indirect calorimeter using a data entry mechanism such as a keypad data port, cable, voice recognition circuitry, or other mechanism such as an electronic communication link.

Docking Station For Indirect Calorimeter

A respiratory analysis system (such as an indirect calorimetry system) may comprise a hand held unit, through which the person breathes during a respiratory test (such as metabolic rate determination, spirometry, or other respiratory analysis), and a docking station. The hand-held unit can comprise a respiratory connector, such as a mask or mouthpiece, a flow path in fluid connection with the respiratory connector, a flow sensor responsive to flow of gas within the flow path, and one or more gas component sensors responsive to the concentration of gas components within the flow path, (such as sensors for oxygen, carbon dioxide, or other respiratory components), a processor for calculating inhaled, exhaled, consumed, and produced gas volumes, a memory, a clock, and a transmitter circuit for transmitting data to the docking station over a cable or wireless link. The docking station may be a self-contained device or an accessory for another device such as a computer. The electronic apparatus referred to in examples above may take the form of a docking station, or combination of computer and docking station. A self-contained device may for example, rest on a surface or be wall mounted, have a power supply, a processor and associated circuitry for analyzing respiratory data, a clock, a cradle for holding the hand-held unit (for example, similar to those used to hold wireless phones), an electrical interface which contacts an interface on the hand-held unit when that unit is placed in the cradle, a memory, a wireless transceiver for receiving data from the hand-held unit during a respiratory test, a wireless Internet connection, and a token reader. The docking station may also have a display for showing the metabolic rate of the person, during or after a test, and mechanisms for providing feedback to the person during a test, for example using methods described in Int. App. WO0213679A2 (“Achieving a relaxed state”), incorporated herein by reference.

A docking station may provide one or more of the following functions: power supply to the hand held unit, recharging a dedicated power supply (such as a battery) in the hand-held unit, transmitting a signal to the hand-held unit allowing it to be used (for example, after the person has used a token to identify themselves to the docking station), receiving data from the hand-held unit (for example by a cable, wireless transmission, or other interface, during and/or after a respiratory test is in progress), analysis of data provided by the hand-held unit, receiving and analyzing data from other sensors (such as cardiac sensors, body temperature sensors, other physiological sensors), communication with a remote computer over a communications network (for example, to transmit measurement data or metabolic data to a remote database, to receive authorization for an identified person to perform a metabolic test), and the like.

A docking station may comprise a token reader or be otherwise adapted to receive an authorization code for use of the hand-held unit. A token reader may comprise a card reader, radio receiver, radio transceiver, IR receiver, electrical connection or interface, capacitive coupling system, inductive coupling system, transponder system, bar code reader, optical sensor, imaging sensor, voice recognition system (e.g. to receive spoken authorization codes), data receiver (e.g. to receive codes from a PDA or other device carried by the person), memory module slot, and the like. For example, a person may insert a card into a card reader associated with the docking station, enter a code into a keypad, or provide a bar code for scanning. The docking station may comprise circuitry for identifying the user from a token, biometric data such as a fingerprint, or other data.

For example, in a fitness center, the docking station may mechanically support a hand-held unit of a respiratory analysis system. A person uses a token to authorize use of the respiratory analyzer, removes the hand-held unit, and breathes through the hand-held unit so as to determine metabolic rate or other physiological data. If the person has pre-purchased a certain number of uses, a successful use of the system reduces the number authorized by one use.

Hence, an improved weight control program comprises the provision of an identity card or other token to a person; association of the identity card with an account; payment to the account by the person; use at intervals of an indirect calorimeter by the person, wherein the use of the indirect calorimeter is authorized by the identity card; and deduction of a monetary value from the account based on a successful metabolic rate measurement using the indirect calorimeter. The identity token may be a card or similar having an identifying mechanism such as a bar code, magnetic stripe, or other mechanism, a key chain, dongle, authorization code (for example, carried by the person or stored on an electronic apparatus such as a communication device, computing device, organizer, electronic key, an the like), wireless transmitter, and the like. The deduction of a monetary value can be related to the charge for the disposable used.

In other embodiments, the counter number can increase with each successful operation of a device, and an authorization code can be used to decrease the counter number (i.e., the increment number is negative). However, this is similar in function to other described embodiments. Hence, the term decrement, as applied to changes in the counter number after each successful operation of the device, can refer generally to a change in counter number value which tends to reduced the usefulness of a device, for example by causing the counter number to approach a predetermined value at (or close to) which the device becomes no longer operational.

The device may store previously used authorization codes in memory, so that attempts by the user to re-use an authorization code may be detected and prevented.

Examples given are not intended to be limiting. Elements of described examples may be combined into other configurations. Other embodiments of the invention will be clear to those skilled in the arts.

Having described our invention, we claim:

1. A device, comprising:
an electrical circuit, comprising a processor, a memory, and a clock, operable to determine a physiological parameter of a user from a physiological signal;
a counter, having a count number decremented by an operation of the electrical circuit to determine the physiological parameter; and
a software program, executable by the processor, operable to receive an authorization code, to determine an increment number from the authorization code, and to change the count number by the increment number, wherein the device can be used a greater number of times by the user after receipt of the authorization code.

2. The device of claim 1, wherein the software program is further operable to prevent operation of the device if the count number is less than a predetermined value.

3. The device of claim 1, wherein the device further comprises a data communications device, providing a communications links with an electronic apparatus, wherein the authorization code is received from the electronic apparatus.

4. The device of claim 3, wherein the electronic apparatus comprises a computer.

5. The device of claim 3, wherein the electronic apparatus comprises a wireless telephone.

6. The device of claim 1, wherein the device further comprises a data communications device, providing a communications link with a communications network, wherein the authorization code is received from a remote computer over the communications network.

7. The device of claim 1, further comprising a data input mechanism whereby a user of the device can enter an authorization code into the device.

8. The device of claim 7, wherein the data input mechanism comprises a plurality of user operable keys.

9. The device of claim 1, wherein the physiological parameter is a metabolic rate of a user, and the physiological signal is correlated with the metabolic rate of the user.

10. The device of claim 9, wherein the physiological signal comprises an oxygen consumption signal correlated with an oxygen consumption of the user.

11. The device of claim 9, wherein the physiological signal comprises a carbon dioxide production signal correlated with a carbon dioxide production of the user.

12. A device, comprising:
a flow path, through which a subject breathes;
a respiratory gas analyzer, providing gas analysis data at least containing flow rate data correlated with a flow rate of respired gases through the flow path;
electrical circuitry, comprising a processor, a memory, and a clock, receiving the gas analysis data and operable to provide a determination of a metabolic parameter of the subject;
a data input mechanism, operable to receive an authorization code; and
a software program, executable by the processor, operable to receive the authorization code, and to enable operation of the device a number of times, the number of times being contained within the authorization code.

13. The device of claim 12, wherein the respiratory gas analyzer comprises a flow rate meter.

14. The device of claim 12, wherein the respiratory gas analyzer comprises an oxygen sensor.

15. The device of claim 12, wherein the respiratory gas analyzer comprises a carbon dioxide sensor.

16. The device of claim 12, wherein the data input mechanism comprises a keypad.

17. The device of claim 12, wherein the data input mechanism comprises a communications link to an electronic apparatus.

18. The device of claim 12, wherein the data input mechanism comprises a communications link to a communications network.

19. The device of claim 12, wherein the metabolic parameter is a metabolic rate of the subject.

20. The device of claim 12, wherein the metabolic parameter is an oxygen consumption of the subject.

21. A system for providing a service to a subject, the system comprising:
a device, comprising a processor and a memory, operable to determine a health-related parameter of the subject;
a communications link between the device and a computer;
computer software, running on the computer, operable to receive identifying data, to generate an authorization code, and to transmit the authorization code to the device over the communications link; and
device software, executed by the processor within the device, operable to receive the authorization code from the computer over the communications link, and to enable one or more operations of the device after receipt of the authorization code.

22. The device of claim 21, wherein the computer is a remote computer associated with a health services provider.

23. The system of claim 21, wherein the health-related parameter is the metabolic rate of the subject.

24. The system of claim 21, wherein the health-related parameter is the oxygen consumption of the subject.

25. The system of claim 21, wherein the communications link comprises a communications network.

26. The system of claim 21, wherein the communications link comprises a wireless link.

27. The system of claim 21, wherein the identifying data comprise a serial number of the device.

28. A method for obtaining at least one operation of a medical device, the medical device providing a health-related service to a subject, the method comprising:
providing an identifier to a supplier of the medical device;
receiving an authorization code from the supplier of the medical device;
providing the authorization code to the medical device; and
operating the respiratory analyzer a number of times, the number of times being encoded within the authorization code, and the number of times being at least one.

29. The method of claim 28, wherein the identifier is a serial number of the medical device.

30. The method of claim 28, wherein the identifier is an identification of the subject.

31. The method of claim 28, wherein the health-related service is the determination of a metabolic rate of the subject.

32. The method of claim 28, wherein providing the identifier to the supplier of the medical device comprises transmitting the identifier to a computer associated with the supplier of the medical device over a communications network.

33. The method of claim 28, wherein receiving the authorization code from the supplier of the medical device comprises receiving the authorization code over a communications network.

34. The method of claim 28, further comprising:
decrypting the authorization code so as to determine an increment number;
incrementing a count number associated with the medical device by the increment number;
decrementing the count number on each successful operation of the device; and
disabling the device if the count number falls below a predetermined value.

35. A method of obtaining a health-related service using a medical device, comprising:
purchasing an authorization code;
providing the authorization code to the medical device, wherein a count number associated with the medical device is incremented by an increment number contained by the authorization code;
operating the medical device, so as to obtain the health-related service, wherein the count number is decremented each time the medical device is operated, and wherein the device does not operate after the count number falls to a predetermined value.

36. The method of claim 35, wherein the medical device is an indirect calorimeter, and the health-related service is a determination of a metabolic rate using the indirect calorimeter.

37. The method of claim 35, wherein the count number is only decremented after a successful operation of the device.

38. The method of claim 35, wherein purchasing an authorization code comprises:
communicating with a remote computer over a communications network;
providing an identifier to the computer; and
receiving the authorization code from the computer over the communications network.

39. The method of claim 35, wherein purchasing the authorization code comprises providing financial information to a seller of the authorization code.

40. The method of claim 35, wherein providing the authorization code to the medical device comprises entering the code into a numeric keypad in communication with the medical device.

41. The method of claim 35, wherein providing the authorization code to the medical device comprises connecting the medical device to an electronic apparatus.

42. The method of claim 35, wherein providing the authorization code to the medical device comprises storing the authorization code on a token, and providing the token to a token reader in communication with the medical device.
